(19) United States
(12) Patent Application Publication
(10) Pub. No.: US 2020/0207793 A1
(43) Pub. Date: Jul. 2, 2020

(54) METHODS FOR IMPROVING PURITY OF TENOFOVIR DISOPROXIL FUMARATE, AND COMPOSITIONS THEREOF

(71) Applicant: AMPAC Fine Chemicals LLC, Rancho Cordova, CA (US)

(72) Inventors: John JACOBSEN, Carmichael, CA (US); Valerie CALDWELL, Shingle Springs, CA (US); Chris DICUS, Davis, CA (US); Patrick BERGET, Roseville, CA (US)

(21) Appl. No.: 16/234,901

(22) Filed: Dec. 28, 2018

(51) Int. Cl. C07F 9/6561; G01N 33/94

(52) U.S. Cl. CPC C07F 9/65616; B01D 3/36; G01N 33/94

(57) ABSTRACT

Methods for producing tenofovir disoproxil fumarate with improved purity are provided. In particular, methods for producing tenofovir disoproxil fumarate with reduced levels of chloromethyl isopropyl carbonate are described. Also described are compositions containing tenofovir disoproxil fumarate with improved purity, and an analysis method that can be used to determine the purity of such compositions with improved accuracy and sensitivity.

### FIELD OF THE INVENTION

The invention relates to methods for producing tenofovir disoproxil fumarate with improved purity, and compositions thereof. In particular, the invention relates to methods for producing tenofovir disoproxil fumarate with a lower content of chloromethyl isopropyl carbonate (CMIC), and compositions thereof.

### BACKGROUND OF THE INVENTION

Tenofovir disoproxil fumarate (TDF) was previously developed by Gilead Sciences, Inc. under the trade name Viread®. TDF has the chemical name 9-[(R)-2-[[bis[[(isopropoxycarbonyl)oxy]methoxy]phosphoryl]methoxy]propyl]adenine fumarate, and the following chemical structure:

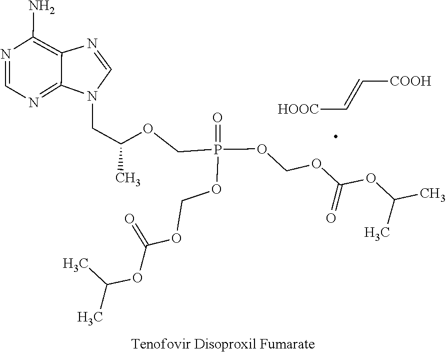 [SMILES: CC(C)OC(=O)OCOP(=O)(CO[C@H](C)Cn1cnc2c(N)ncnc21)OCOC(=O)OC(C)C ; O=C(O)/C=C/C(=O)O]

Tenofovir disoproxil fumarate (TDF) is a single nucleotide phosphate prodrug, which converts to tenofovir, chemically known as 9-[(R)-2-phosphonomethoxypropyl]adenine (PMPA), after oral administration into the body of a subject. Tenofovir is a highly potent antiviral agent, particularly for the therapy and prophylaxis of retroviral infections, and belongs to a class of drugs known as nucleotide reverse transcriptase inhibitor (NRTI).

Tenofovir disoproxil and its pharmaceutically acceptable salts were first disclosed in U.S. Pat. No. 5,922,695. This patent discloses the preparation of tenofovir disoproxil by the esterification of tenofovir with chloromethyl isopropyl carbonate (CMIC) using 1-methyl-2-pyrrolidinone and triethylamine. According to this patent, tenofovir disoproxil was converted to the fumarate salt without isolation.

International Patent Application Publication WO 2008007392 A2 discloses a process for the preparation of tenofovir disoproxil fumarate, wherein the tenofovir disoproxil is also prepared by esterification of tenofovir with CMIC, and the isolated crystalline tenofovir disoproxil is then further converted to the hemifumarate salt.

U.S. Patent Application Publication No. 20130005969 A1 discloses a process for the preparation of tenofovir disoproxil by the esterification of tenofovir with CMIC in the presence of a phase transfer catalyst and a dehydrating agent. The obtained crude tenofovir disoproxil is then converted to the fumarate salt.

A number of other prior art references disclose similar processes for the preparation of tenofovir disoproxil fumarate, which also involve the esterification of tenofovir with chloromethyl isopropyl carbonate (CMIC).

In the known processes for preparing tenofovir disoproxil fumarate, unreacted chloromethyl isopropyl carbonate (CMIC) remains as an impurity in the final product. Furthermore, the amount of CMIC varies widely among different preparations of tenofovir disoproxil fumarate. Therefore, there remains a need for tenofovir disoproxil fumarate preparations having improved purity, particularly with a reduced content of impurities such as CMIC, as well as methods for preparing tenofovir disoproxil fumarate with improved purity, and for accurately determining the purity thereof.

### BRIEF SUMMARY OF THE INVENTION

The invention addresses this need by providing compositions of tenofovir disoproxil fumarate (TDF) with improved purity and methods of preparation thereof, as compared to the purity of TDF compositions obtained by other methods known in the art for preparing TDF. In particular, the invention relates to a composition comprising TDF having a reduced level of chloromethyl isopropyl carbonate (CMIC), and methods of preparing such compositions.

In one general aspect, the invention relates to a composition comprising tenofovir disoproxil fumarate, wherein a content of chloromethyl isopropyl carbonate in the composition, as measured by analyzing a solution of the composition by direct injection gas chromatography (GC), is about 50 ppm or less.

In one particular embodiment, the content of chloromethyl isopropyl carbonate in a composition of the invention is about 25 ppm or less.

In another particular embodiment, the content of chloromethyl isopropyl carbonate in a composition of the invention is about 15 ppm or less.

In another general aspect, the invention relates to a method of preparing a composition comprising tenofovir disoproxil fumarate (TDF), wherein a content of chloromethyl isopropyl carbonate in the composition, as measured by analyzing a solution of the composition by direct injection gas chromatography (GC), is 50 ppm or less, the method comprising:
(i) reacting tenofovir disoproxil fumarate with a base to obtain a first tenofovir disoproxil preparation, or alternatively using a prepared crude preparation of tenofovir disoproxil freebase as a first tenofovir disoproxil preparation;
(ii) washing the first tenofovir disoproxil preparation with water to obtain a first mixture, and drying the first mixture to obtain a second tenofovir disoproxil preparation;
(iii) diluting the second tenofovir disoproxil preparation with an organic solvent to obtain a second mixture, and adding silica to the second mixture to obtain a third mixture;
(iv) removing the silica from the third mixture and concentrating the filtered third mixture to obtain a third tenofovir disoproxil preparation;
(v) reacting the third tenofovir disoproxil preparation with fumaric acid in a solvent to obtain a fourth mixture comprising tenofovir disoproxil fumarate; and
(vi) crystallizing the tenofovir disoproxil fumarate from the fourth mixture to obtain the composition.

In another general aspect, the invention relates to a composition comprising tenofovir disoproxil fumarate, wherein a content of chloromethyl isopropyl carbonate (CMIC) in the composition is abut 50 ppm or less, by analyzing a solution of the composition by direct injection gas chromatography (GC), wherein the composition is prepared by a method of the invention.

In yet another general aspect, the invention relates to a method for quantifying a content of chloromethyl isopropyl carbonate (CMIC) in a composition comprising tenofovir disoproxil fumarate (TDF), the method comprising:
(i) dissolving the composition in a solvent to prepare a sample solution;
(ii) dissolving a reference standard of CMIC in a solvent to prepare a standard solution
(iii) analyzing the sample solution and standard solution by direct injection gas chromatography;
(iv) identifying a peak corresponding to CMIC for each of the sample solution and the standard solution from step (iii); and
(v) quantifying the content of CMIC in the composition by comparing an area of the peak corresponding to CMIC in the sample solution to an area of the peak corresponding to CMIC in the standard solution.

In yet other general aspects, the invention relates to a pharmaceutical composition comprising at least one pharmaceutically acceptable carrier and a composition described herein, and methods of treating or preventing hepatitis B virus (HBV) (e.g., chronic HBV) or human immunodeficiency virus (HIV) in a subject in need thereof with a composition or pharmaceutical composition of the invention as described herein.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a representative blank chromatogram of direct injection gas chromatography (GC).

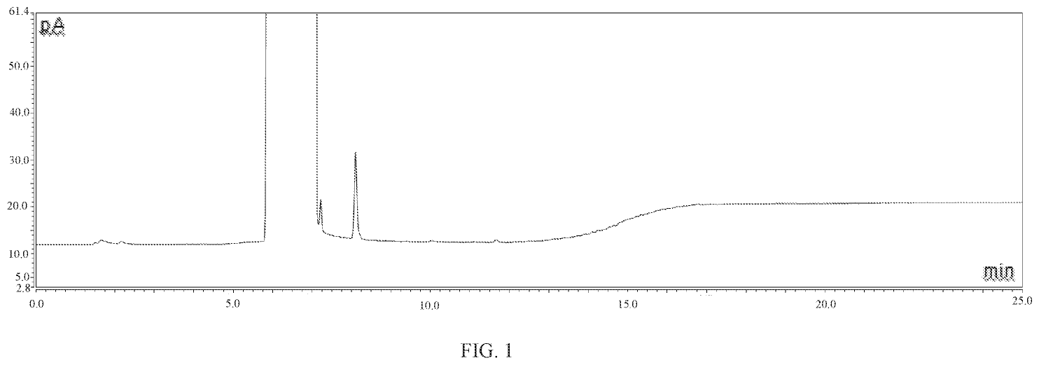

FIG. 2 shows a representative CMIC external standard chromatogram of direct injection gas chromatography (GC), which shows the peak of CMIC at 9.2 minutes.

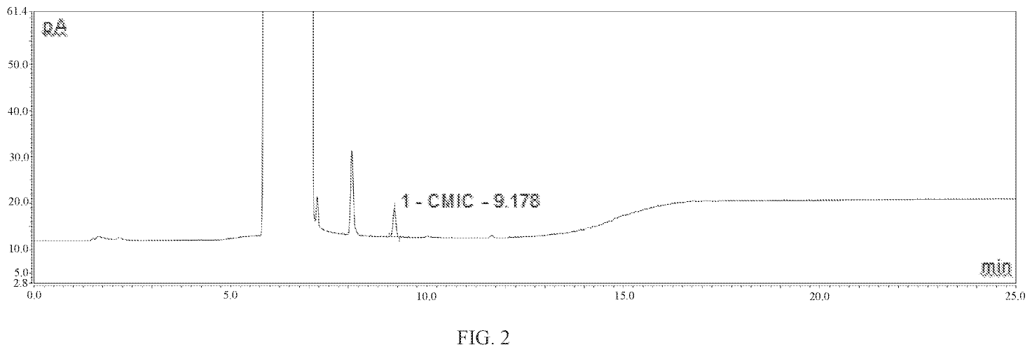

FIG. 3 shows a sample TDF composition chromatogram of direct injection gas chromatography (GC), which contains CMIC as an impurity.

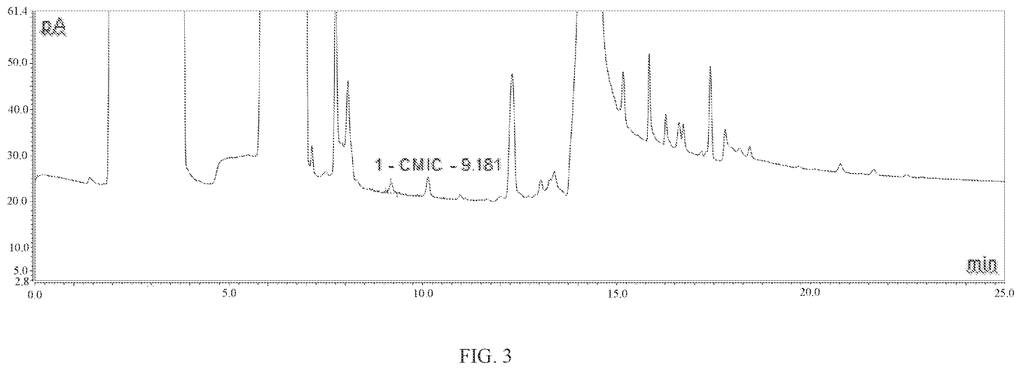

### DETAILED DESCRIPTION OF THE INVENTION

Unless defined otherwise, all technical and scientific terms used herein have the same meaning as is commonly understood by one of ordinary skill in the art to which this invention pertains. Otherwise, certain terms used herein have the meanings as set in the specification. All patents, published patent applications and publications cited herein are incorporated by reference as if set forth fully herein.

All publications and patents referred to herein are incorporated by reference. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

It must be noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, an amount of about 50 ppm or less includes 45 ppm or less to 55 ppm or less. As used herein, the use of a numerical range expressly includes all possible subranges, all individual numerical values within that range, including integers within such ranges and fractions of the values unless the context clearly indicates otherwise.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “ppm” as used with reference to a particular compound refers to parts per million of that compound, which are commonly used as a measure of small levels/concentrations. PPM is a dimensionless quantity, as a ratio of two quantities of the same unit (such as weight, molar, or volume). The basic formula to calculate ppm is to multiple the ratio by 1,000,000. ppm is a value that represents the part of a whole number in units of 1/1000000, for example, 1% equals 10,000 ppm.

Processes for preparing tenofovir disoproxil fumarate usually involve the preparation of an intermediate, tenofovir disoproxil, by esterification of tenofovir with chloromethyl isopropyl carbonate (CMIC), followed by a reaction with fumaric acid to obtain tenofovir disoproxil fumarate, as shown in Scheme 1 below:

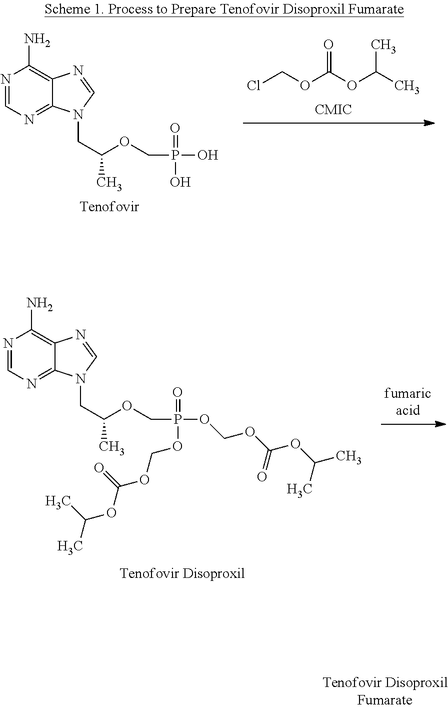

One of the disadvantages of the above-described process is that the resulting tenofovir disoproxil, and thus tenofovir disoproxil fumarate, is obtained with the impurity CMIC present in the final product, which results from unreacted CMIC from the step of esterification. To the best of the knowledge of the inventors, there currently is no process to obtain tenofovir disoproxil preparations, such as tenofovir disoproxil fumarate preparations, having improved purity by reducing the amount or content of CMIC in such preparations. European Chemicals Agency (ECHA) lists chloromethyl isopropyl carbonate (under the synonym chlormethyl-propan-2-ylcarbonate) as a suspected carcinogen and suspected mutagen. However, there is currently no regulatory limit set by, e.g., the US Food and Drug Administration (FDA) for controlling the amount of CMIC in tenofovir disoproxil preparations. As a result, current processes for preparing tenofovir disoproxil and salts thereof, such as tenofovir disoproxil fumarate, result in preparations having varying amounts of CMIC, ranging from about 70-80 ppm to about 3000 ppm and possibly higher.

The inventors of the present invention have thus discovered an effective purification process to reduce the amount of CMIC in tenofovir disoproxil preparations, thus improving the purity of final tenofovir disoproxil products, such as tenofovir disoproxil fumarate.

In one general aspect, the invention relates to a method of preparing a composition comprising tenofovir disoproxil fumarate (TDF). The method described herein effectively removes common impurities in TDF preparations, particularly the impurity CMIC. According to embodiments of the invention, the method provides a composition having a reduced content of chloromethyl isopropyl carbonate (CMIC).

In particular, a method of the invention comprises one or more of the following steps:

(i) reacting tenofovir disoproxil fumarate with a base to obtain a first tenofovir disoproxil preparation, or alternatively using a prepared crude preparation of tenofovir disoproxil freebase as a first tenofovir disoproxil preparation;

(ii) washing the first tenofovir disoproxil preparation with water to obtain a first mixture, and drying the first mixture to obtain a second tenofovir disoproxil preparation;

(iii) diluting the second tenofovir disoproxil preparation with an organic solvent to obtain a second mixture, and adding silica to the second mixture to obtain a third mixture;

(iv) removing the silica from the third mixture and concentrating the filtered third mixture to obtain a third tenofovir disoproxil preparation; and

(v) reacting the third tenofovir disoproxil preparation with fumaric acid in a solvent to obtain a fourth mixture comprising tenofovir disoproxil fumarate.

In some embodiments, the method further comprises crystallizing the tenofovir disoproxil fumarate from the mixture to obtain the composition.

According to embodiments of the invention, tenofovir disoproxil fumarate used in step (i) can be prepared by any method known in the art in view of the present disclosure for synthesizing tenofovir disoproxil and converting tenofovir disoproxil to the fumarate salt. Thus, tenofovir disoproxil fumarate used in step (i) of a method of the invention typically comprises a content of chloromethyl isopropyl carbonate (CMIC) that is higher than 50 ppm. The tenofovir disoproxil fumarate can be crude tenofovir disoproxil fumarate. As used herein, “crude tenofovir disoproxil fumarate” refers to the product from the reaction of tenofovir disoproxil and fumaric acid before any crystallization step. In certain embodiments, the tenofovir disoproxil fumarate in step (i) can also be crystallized tenofovir disoproxil fumarate. Crystallized tenofovir disoproxil and crude tenofovir disoproxil can be obtained by any method known in the art involving the use of CMIC in view of the present disclosure. For example, tenofovir disoproxil can be obtained by esterification of tenofovir with CMIC. Tenofovir disoproxil can be synthesized by any method known in the art in view of the present disclosure. The obtained tenofovir disoproxil can be reacted with fumaric acid to produce crude tenofovir disoproxil fumarate, which can be used directly in the methods of the invention described herein. Alternatively, the obtained tenofovir disoproxil can be reacted with fumaric acid followed by crystallization to produce crystallized tenofovir disoproxil fumarate, which then can be used in the methods of the invention described herein.

In certain embodiments, tenofovir disoproxil fumarate is reacted with a base in a solvent. The solvent is preferably an organic solvent. Examples solvents suitable for use for this purpose include, but are not limited to, acetone, methanol, ethanol, 2-propanol, acetonitrile, methyl acetate, ethyl acetate, isopropyl acetate, dichloromethane, toluene, isopropyl alcohol, diethyl ether, and isopropyl ether. In certain embodiments, the solvent is a mixed solvent of at least one of methyl acetate, ethyl acetate, and isopropyl acetate with water. Preferably, the solvent is isopropyl acetate or a mixture of isopropyl acetate and water.

In certain embodiments, tenofovir disoproxil is reacted with a base. The base can be an inorganic base or an organic base. Examples of organic bases suitable for use in the invention include, but are not limited to, triethylamine, diethylamine, N,N-diisopropylethylamine, aminomethyl propanol, and ethanolamine. Examples of inorganic bases suitable for use in the invention include, but are not limited to, sodium hydroxide, potassium hydroxide, sodium carbonate, sodium bicarbonate, potassium carbonate, potassium bicarbonate, cesium carbonate, cesium bicarbonate, ammonium hydroxide, and ammonium acetate. Preferably, the base is ethanolamine, sodium bicarbonate, or potassium bicarbonate. In some embodiments, the mole ratio of the base to TDF is 1 to 4 molar equivalents of base relative to TDF, such as 1, 2, 3, or 4 molar equivalents of base relative to TDF, and is preferably at least 2 molar equivalents, and more preferably is 3 molar equivalents.

In preferred embodiments, when the base is an organic base, the solvent used is a mixture of isopropyl acetate and water. In another preferred embodiment, when the base is an inorganic base, the solvent used is isopropyl acetate and the inorganic base is used as an aqueous solution, for instance aqueous sodium bicarbonate.

According to embodiments of the invention, reaction of tenofovir disoproxil fumarate with a base, preferably in a solvent, produces a mixture comprising tenofovir disoproxil (free base). In certain embodiments, the mixture is a biphasic mixture, which can be separated to obtain the organic phase as the first tenofovir disoproxil preparation. In certain embodiments, the aqueous phase can be extracted with an organic solvent and combined with the organic phase to produce the first tenofovir disoproxil preparation.

In certain embodiments, a prepared crude preparation of tenofovir disoproxil freebase is alternatively used as a first tenofovir disoproxil preparation.

According to embodiments of the invention, the first tenofovir disoproxil preparation comprises tenofovir disoproxil (free base) and a reduced content of chloromethyl isopropyl carbonate. This tenofovir disoproxil preparation is then washed with water to produce a first mixture. In certain embodiments, the washing with water in the step (ii) can be repeated. This washing procedure results in a mixture, which comprises residual water. The residual water can lead to the formation of the monoPOC impurity over time (shown below). It is thus preferable to remove the residual water for the purpose of purification. Residual water can be removed from this mixture by drying the mixture, such as by using a drying agent or by azeotropic distillation.

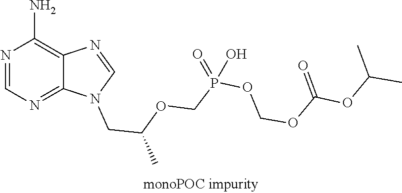 [SMILES: CC(C)OC(=O)OCOP(=O)(O)CO[C@H](C)Cn1cnc2c(N)ncnc21]

In some embodiments, the mixture comprising tenofovir disoproxil (free base) is dried after washing to produce a second tenofovir disoproxil tenofovir disoproxil preparation by using a drying agent, such as sodium sulfate, magnesium sulfate, or molecular sieves.

In some embodiments, the mixture comprising tenofovir disoproxil (free base) is dried after washing to produce a second tenofovir disoproxil preparation by azeotropic distillation. Azeotropic distillation is an effective method to remove residual water by concentration of a solution of the compounds from an appropriate solvent. As used herein, azeotropic distillation refers in particular to the concentration of a mixture comprising tenofovir disoproxil (free base) to remove residual water after washing of the mixture following base treatment in a solvent. In certain embodiments, additional solvent is added prior to the concentration of the mixture. The additional solvent is selected from the group consisting of solvents including, but not limited to, methanol, ethanol, isopropanol, and dimethylformamide. Preferably, the additional solvent is dimethylformamide (DMF) or isopropyl alcohol (IPA). The addition of such a solvent, e.g., DMF or IPA, prior to azeotropic distillation can prevent crystallization of tenofovir disoproxil during distillation and aid in solubility during subsequent purification steps.

According to embodiments of the invention, the second tenofovir disoproxil preparation obtained after drying (e.g., with a drying agent or by azeotropic distillation), is added with a solvent to form a second mixture. This mixture preferably has a reduced water content as a result of the prior drying step(s). Examples of solvents suitable for use for this purpose include, but are not limited to, acetone, methanol, ethanol, 2-propanol, acetonitrile, methyl acetate, ethyl acetate, isopropyl acetate, dichloromethane, isopropyl alcohol, diethyl ether, and isopropyl ether. Preferably, the solvent is isopropyl acetate or a mixture of isopropyl acetate and isopropyl alcohol. This mixture is subsequently converted to a second tenofovir disoproxil preparation. In certain embodiments, this mixture is converted to the third tenofovir disoproxil preparation by direct concentration. The concentration can be done according to any method known in the art in view of the present disclosure, e.g., under vacuum. In other embodiments, the mixture is treated with silica.

According to embodiments of the invention, treatment with silica can further reduce the amount of chloromethyl isopropyl carbonate in the tenofovir disoproxil preparation. The silica that can be used includes, but is not limited to, standard silica and thiol modified silica. Standard silica is also known as silicon dioxide (SiO₂), silicic acid or silicic acid anhydride. Silica can be obtained from dehydration of orthosilicic acid (Si(OH)₄). Silica is widely used for separation and purification of compounds. As used herein, “thiol-modified silica” or “Si-thiol” refers to silica functionalized with multiple thiol (—SH) groups. Thiol-modified silica thus contains silica particles with a surface functionalized with thiol group (—SH). Both standard silica and thiol-modified silica can absorb CMIC.

Standard silica or thiol-modified silica can be added to a mixture containing tenofovir disoproxil in a solvent, and the resulting mixture can be stirred for a suitable period of time, either at room temperature or an elevated temperature for instance at temperature of 25° C. to 50° C. In some embodiments, the loading of standard silica or thiol-modified silica is about 40 wt % to 60 wt %, for instance about 50 wt % relative to the amount of charged TDF. The mixture can be stirred for about 30 minutes to 4 hours, such as 30 minutes, 1 hour, 2 hours, 3 hours, or 4 hours, preferably about 2 hours. Then, the mixture can be filtered and the resulting filtrate can be concentrated to produce a third tenofovir disoproxil preparation.

According to embodiments of the invention, a method further comprises reacting the third tenofovir disoproxil preparation with fumaric acid in a solvent to obtain a fourth mixture comprising tenofovir disoproxil fumarate; and crystallizing the tenofovir disoproxil fumarate from the mixture to obtain a composition comprising TDF, preferably wherein the composition has a reduced content of CMIC, e.g., about 50 ppm or less.

Tenofovir disoproxil fumarate can be obtained by reacting tenofovir disoproxil and fumaric acid according to any method known in the art in view of the present disclosure. In some embodiments, tenofovir disoproxil is reacted with fumaric acid in a solvent. For example, a mixture of tenofovir disoproxil in a solvent can be heated, e.g., to a temperature of about 40° C. to 60° C., for instance about 50° C., until substantially all of the tenofovir disoproxil is dissolved. Then fumaric acid can be added and further heated to a temperature of about 60° C. to 80° C., for instance about 70° C., until a homogeneous solution is provided. Examples of solvents suitable for use in the invention for this purpose include, but are not limited to methanol, ethanol, propanol, and isopropyl alcohol. Once the reaction with fumaric acid is complete, tenofovir disoproxil fumarate can be recovered from the reaction mixture. Methods for recovering the compound from the reaction mixture are not particularly limited, and any method known in the art can be used to isolate tenofovir disoproxil fumarate, such as distillation, filtration, crystallization, precipitation, etc. In a preferred embodiment, tenofovir disoproxil fumarate is isolated from the solution by crystallization. For example, after salt formation at elevated temperature, the reaction mixture can be slowly cooled down to room temperature or lowered to precipitate out the formed solid. The precipitated solid can be filtered, washed and dried. In a particular embodiment, after a homogeneous solution is provided upon heating, the solution can be cooled, e.g., to about 30° to 50° C., and seeded with a small amount of tenofovir disoproxil fumarate induce crystallization of tenofovir disoproxil fumarate from the solution. One of ordinary skill in the art will readily be able to determine and employ the appropriate techniques for recovering tenofovir disoproxil fumarate from the solution after salt formation in order to maximize compound yield, purity, etc.

Further purification of tenofovir disoproxil fumarate can be achieved by recrystallization. Any water soluble organic solvent can be used for recrystallization, including, but not limited to, acetonitrile, acetone and other water soluble ketones, water soluble alcohols, THF and other water soluble ethers, diglyme and other glymes, and mixtures of the same. Examples of suitable alcohols include methyl alcohol, ethyl alcohol, n-propyl alcohol, n-butyl alcohol, iso-butyl alcohol, tertiary butyl alcohol, n-pentyl alcohol, iso-pentyl alcohol, and neo-pentyl alcohol.

A method of preparing a composition comprising tenofovir disoproxil fumarate having a reduced content of chloromethyl isopropyl carbonate according to an embodiment of the present invention is depicted in Scheme 2 below.

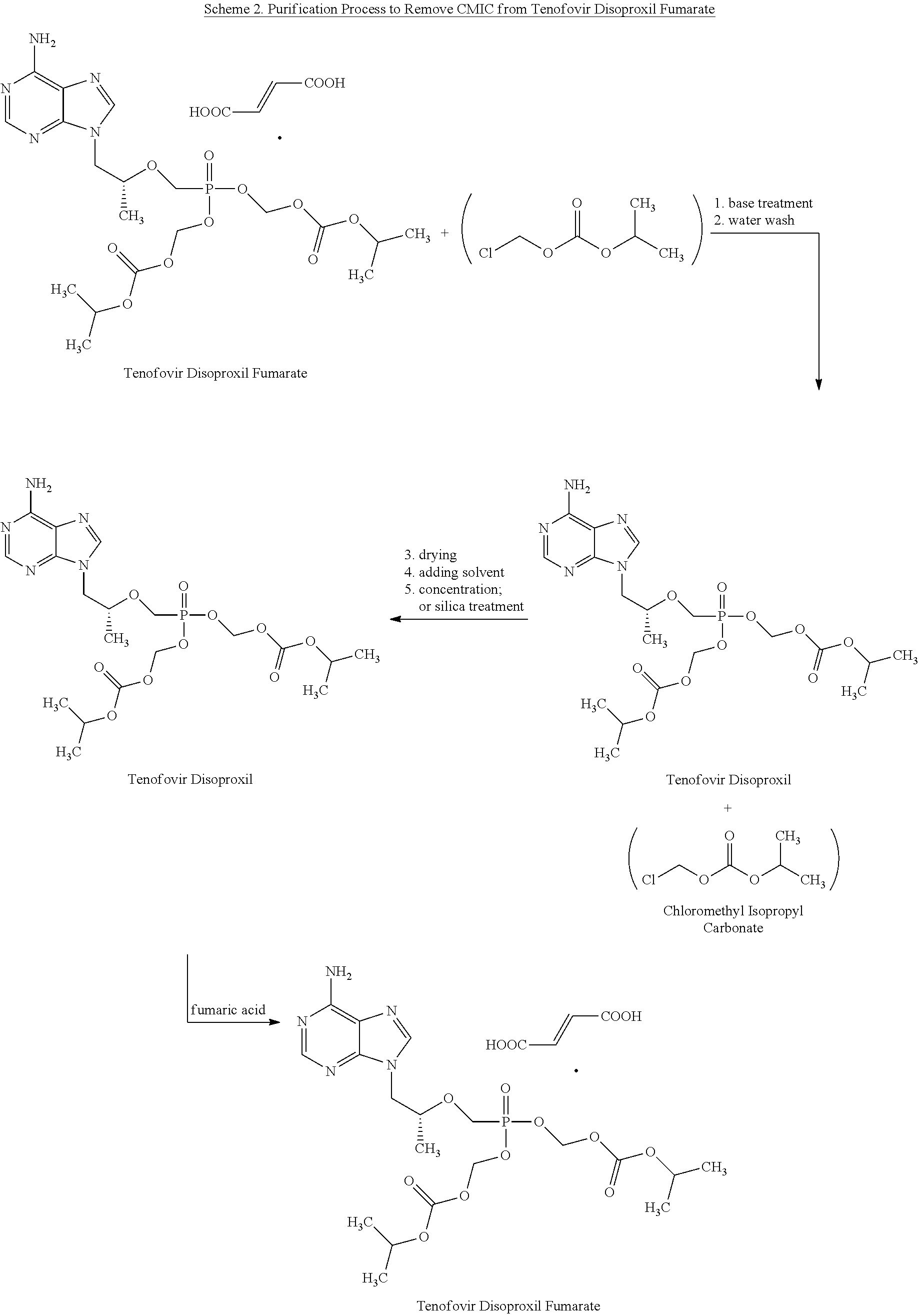

In yet another general aspect, the invention relates to a composition comprising tenofovir disoproxil fumarate. According to embodiments of the invention, a content of chloromethyl isopropyl carbonate in a composition of the invention is about 50 ppm or less. Such compositions can be prepared by any of the methods described herein. Preferably, the CMIC is present in a reduced amount as compared to the amount of CMIC in TDF compositions prepared according to other methods known in the art.

In some embodiments, a content of CMIC in a composition of the invention is about 50 ppm or less, such as 45 ppm or less, 40 ppm or less, 35 ppm or less, 30 ppm or less, 25 ppm or less, 20 ppm or less, 15 ppm or less, or 10 ppm or less, or any value in between, preferably about 25 ppm or less, and more preferably about 10 ppm or less. The amount of CMIC in a composition of the invention can be determined by any method known in the art in view of the present disclosure, such as by gas chromatography, e.g., headspace gas chromatography (HSGC), direct injection gas chromatography, etc.

In some embodiments, a content of CMIC in a composition of the invention is measured by analyzing a solution of the composition by gas chromatography, preferably direct injection gas chromatography. Gas chromatography is a common type of chromatography used in analytical chemistry for separating and analyzing compounds that can be vaporized without decomposition. Typically, CMIC content in TDF preparations has been determined byhead-space gas chromatography (HSGC). Such methods are described in the USP Pending Monograph (version 1, authorized Sep. 1, 2011), which describes HSGC analysis of CMIC on 100% dimethylpolysiloxane column at 1500 ppm. However, such gas chromatography methods did not provide adequate sensitivity for trace analysis of residual amounts of CMIC. Accordingly, the inventors developed a method for determining the content of CMIC in TDF preparations that provides for improved sensitivity in detecting and quantifying the amount of CMIC in such preparations, e.g., for detecting CMIC content of about 50 ppm or less, preferably about 25 ppm or less, more preferably about 10 ppm or less. In particular, the inventors found that by analyzing a solution of a composition of the invention by gas chromatography, particularly direct injection gas chromatography, the sensitivity of CMIC detection can be improved and the amount of CMIC thus more accurately quantified, as compared to other methods of analysis, such as headspace gas chromatography.

In one embodiment, a content of CMIC in a composition of the invention is measured by analyzing a solution of the composition by direct injection gas chromatography. A solution of the composition can be prepared in any solvent, such as an organic solvent, suitable for use in gas chromatography. Such solution is referred to as a “sample solution.” Examples of solvents suitable for use in the invention for this purpose include, but are not limited to, dimethylsulfoxide (DMSO), dimethylacetamide (DMAc), 1,3-dimethyl-2-imidazolidinone (DMI), dimethylformamide (DMF), N-methyl-2-pyrrolidone (NMP), N,N′-dimethylpropyleneurea (DMPU), and hexamethylphosphoramide (HMPA). In a preferred embodiment, the solvent is DMF. In some embodiments, the direct injection gas chromatography is high load direct injection gas chromatography.

According to embodiments of the invention, a sample solution for analysis by direct injection gas chromatography can be prepared from a composition of the invention, such that the concentration of TDF in the solution is about 100 mg/mL to 300 mg/mL, such as 100 mg/mL, 120 mg/mL, 140 mg/mL, 160 mg/mL, 180 mg/mL, 200 mg/mL, 220 mg/mL, 240 mg/mL, 260 mg/mL, 280 mg/mL, or 300 mg/mL, preferably about 180 mg/mL to 200 mg/mL, for instance about 200 mg/mL. For example, a sample solution of a composition of the invention for analysis by direct injection gas chromatography can be prepared at concentration of about 200 mg/mL TDF in DMF.

Once a sample solution of a composition of the invention is prepared, the sample solution is analyzed by direct injection gas chromatography. In some embodiments, a capillary gas chromatography column is used, which is a narrow tube in which the stationary phase coats the interior surface of the column. In a particular embodiment, a 95% dimethyl/5% diphenyl polysiloxane or 6% cyanopropylphenyl/94% dimethylpolysiloxane capillary gas chromatography column is used. The inlet temperature should be optimized so as to exceed the boiling point of CMIC, which is about 147° C. Thus, in particular embodiments, the inlet temperature is about 150° C. to 170° C., such as about 150° C., 155° C., 160° C., 165° C., or 170° C., or any value in between, preferably about 160° C.

Any method of detection known in the art in view of the present disclosure can be used, such as flame ionization detection, electrochemical detection, mass spectrometric detection, etc. Preferably, the detection method comprises flame ionization detection.

According to embodiments of the invention, quantitative analysis of CMIC is performed using an external standard of CMIC. A reference solution of CMIC can be prepared by dissolving a standard of CMIC in any solvent, such as an organic solvent, suitable for use in gas chromatography. Examples of solvents suitable for use in the invention for this purpose include, but are not limited to, dimethylsulfoxide (DMSO), dimethylacetamide (DMAc), 1,3-dimethyl-2-imidazolidinone (DMI), dimethylformamide (DMF), N-methyl-2-pyrrolidone (NMP), N,N′-dimethylpropyleneurea (DMPU), and hexamethylphosphoramide (HMPA). Preferably, the same solvent is used to prepare both the sample solution of the composition and the reference solution of CMIC. In a preferred embodiment, the solvent used to prepare both the sample solution of the composition and the reference solution of CMIC is DMF.

Thus, in another general aspect, the invention relates to a method of quantifying a content of CMIC in a composition comprising TDF, such as a composition of the invention. According to embodiments of the invention, the method comprises:

(i) dissolving the composition in a solvent to prepare a sample solution;

(ii) dissolving a reference standard of CMIC in a solvent to prepare a standard solution

(iii) analyzing the sample solution and standard solution by direct injection gas chromatography;

(iv) identifying a peak corresponding to CMIC for each of the sample solution and the standard solution from step (iii); and

(v) quantifying the content of CMIC in the composition by comparing an area of the peak corresponding to CMIC in the sample solution to an area of the peak corresponding to CMIC in the standard solution.

According to embodiments of the invention, the resulting peak area of the CMIC in the TDF sample solution is used to calculate the CMIC content in ppm based on the response relative to an external standard of CMIC. Specifically, the CMIC content (ppm) can be calculated as follows:

$$CMIC ( ppm ) = \frac{{A}_{Sam}}{{A}_{Std}} \times \frac{{C}_{Std}}{{C}_{Sam}} \times 1000000$$

where:
Aₛₐₘ: peak area of CMIC in the sample solution
A_{std}: average peak area of CMIC in the Standard Solution
C_{Sam}: Sample Concentration (mg/mL)
C_{Std}: Working Standard CMIC Concentration (mg/mL)

In some embodiments, a method of preparing a composition comprising tenofovir disoproxil fumarate of the invention further comprises quantifying or determining a content or amount of CMIC in the composition. Preferably, the content or amount of CMIC is quantified or determined by analyzing a solution of the composition by gas chromatography according to a method of the invention.

In another general aspect, the invention relates to a composition comprising TDF produced by a method of the invention. Compositions comprising TDF prepared according to the method described herein can further comprise CMIC. In preferred embodiments, a TDF composition comprises a reduced amount of CMIC, meaning that the amount of CMIC present in the composition relative to the composition is about 50 ppm or less, preferably about 25 ppm or less, and more preferably about 10 ppm or less.

In a particular embodiment, a composition comprising tenofovir disoproxil fumarate is obtained by a method comprising:

(i) reacting tenofovir disoproxil fumarate with a base to obtain a first tenofovir disoproxil preparation, or alternatively using a prepared crude preparation of tenofovir disoproxil freebase as a first tenofovir disoproxil preparation;

(ii) washing the first tenofovir disoproxil preparation with water to obtain a first mixture, and drying the first mixture to obtain a second tenofovir disoproxil preparation;

(iii) diluting the second tenofovir disoproxil preparation with an organic solvent to obtain a second mixture, and adding silica to the second mixture to obtain a third mixture;

(iv) removing the silica from the third mixture and concentrating the filtered third mixture to obtain a third tenofovir disoproxil preparation;

(v) reacting the third tenofovir disoproxil preparation with fumaric acid in a solvent to obtain a fourth mixture comprising tenofovir disoproxil fumarate; and

(vi) crystallizing the tenofovir disoproxil fumarate from the fourth mixture to obtain the composition.

The composition can further comprise CMIC in an amount that is about 50 ppm or less, preferably about 25 ppm or less, more preferably about 10 ppm or less, as measured by analyzing a solution of the composition by gas chromatography.

In another aspect, the invention relates to a method of treating or preventing a viral infection in a subject, such as hepatitis B virus (HBV) (e.g., chronic HBV) or human immunodeficiency virus (HIV) in a subject in need thereof. According to embodiments of the invention, such method comprises administering to the subject a composition comprising tenofovir disoproxil fumarate as described herein. Preferably, a subject is a mammal, more preferably a human subject.

In some embodiments, a composition administered to a subject is a pharmaceutical composition further comprising at least one pharmaceutically acceptable carrier. A “carrier” refers to any excipient, diluent, buffer, stabilizer, or other material well known in the art for pharmaceutical formulations. Pharmaceutically acceptable carriers in particular are non-toxic and should not interfere with the efficacy of the active ingredient. Pharmaceutically acceptable carriers can be readily determined by one of ordinary skill in the art, and include excipients and/or additives suitable for use in the pharmaceutical compositions known in the art, e.g., as listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are entirely incorporated herein by reference.

### Embodiments of the Invention

The invention also provides the following non-limiting embodiments.

Embodiment 1 is a composition comprising tenofovir disoproxil fumarate and optionally further comprising chloromethyl isopropyl carbonate.

Embodiment 1a is the composition of embodiment 1, wherein a content of chloromethyl isopropyl carbonate in the composition is 50 ppm or less, as measured by gas chromatography (GC).

Embodiment 1b is the composition of embodiment 1, wherein a content of chloromethyl isopropyl carbonate in the composition is 25 ppm or less, as measured by gas chromatography (GC).

Embodiment 1c is the composition of embodiment 1, wherein a content of chloromethyl isopropyl carbonate in the composition is 10 ppm or less, as measured by gas chromatography (GC).

Embodiment 1d is the composition of any one of embodiments 1-1c, wherein the content of chloromethyl isopropyl carbonate in the composition is determined by analyzing a solution of the composition by direct inject gas chromatography.

Embodiment 2 is a pharmaceutical formulation comprising at least one pharmaceutically acceptable excipient and the composition of any one of embodiments 1-1d.

Embodiment 3 is a method of treating or preventing chronic hepatitis B in a subject in need thereof, the method comprising administering to the subject the composition of any one of embodiments 1-1d or the pharmaceutical composition of embodiment 2.

Embodiment 3a is a method of treating or preventing human immunodeficiency virus (HIV) in a subject in need thereof, the method comprising administering to the subject the composition of any one of embodiments 1-1d or the pharmaceutical composition of embodiment 2.

Embodiment 4 is a method of preparing a composition comprising tenofovir disoproxil fumarate, the method comprising:

(i) reacting tenofovir disoproxil fumarate in a solvent with a base to obtain a first tenofovir disoproxil preparation, or alternatively using a prepared crude preparation of tenofovir disoproxil freebase as a first tenofovir disoproxil preparation; and

(ii) washing the first tenofovir disoproxil preparation with water to obtain a first mixture.

Embodiment 4a is the method of embodiment 4, wherein a content of chloromethyl isopropyl carbonate in the composition is 50 ppm or less, as measured by gas chromatography

(GC).

Embodiment 4b is the method of embodiment 4, wherein a content of chloromethyl isopropyl carbonate in the composition is 25 ppm or less, as measured by gas chromatography (GC).

Embodiment 4c is the method of embodiment 4, wherein a content of chloromethyl isopropyl carbonate in the composition is 10 ppm or less, as measured by gas chromatography (GC).

Embodiment 4d is the method of any one of embodiments 4-4c, wherein a content of chloromethyl isopropyl carbonate is determined by analyzing a solution of the composition by direct injection gas chromatography (GC).

Embodiment 5 is the method of any one of embodiments 4-4d, wherein the solvent used in the step (i) is selected from the group consisting of acetone, methanol, ethanol, 2-propanol, acetonitrile, methyl acetate, ethyl acetate, isopropyl acetate, dichloromethane, toluene, isopropyl ether, diethyl ether, and isopropyl ether.

Embodiment 5a is the method of any one of embodiments 4-4d, wherein the solvent used in the step (i) is a mixed solvent of at least one of methyl acetate, ethyl acetate, and isopropyl acetate with water.

Embodiment 5b is the method of any one of embodiments 4-4d, wherein the solvent used in the step (i) is isopropyl acetate or a mixture of isopropyl acetate and water.

Embodiment 6 is the method of any one of embodiments 4-5b, wherein the base is an organic base, preferably selected from the group consisting of triethylamine, diethylamine, N,N-diisopropylethylamine, aminomethyl propanol, and ethanolamine.

Embodiment 6a is the method of any one of embodiments 4-5b, wherein the base is an inorganic base, preferably selected from the group consisting sodium hydroxide, potassium hydroxide, sodium carbonate, sodium bicarbonate, potassium carbonate, potassium bicarbonate, cesium carbonate, cesium bicarbonate, ammonium hydroxide, and ammonium acetate.

Embodiment 6b is the method of embodiment 6, wherein the base is ethanolamine.

Embodiment 6c is the method of embodiment 6a, wherein the base is sodium bicarbonate or potassium bicarbonate.

Embodiment 7 is the method of any one of embodiments 4-6c, further comprising:
(iii) drying the first mixture from the step (ii) to obtain a second tenofovir disoproxil preparation;
(iv) adding a solvent to the second tenofovir disoproxil preparation to obtain a second mixture; and
(v) concentrating the second mixture from the step (iv) to obtain a third tenofovir disoproxil preparation; or treating the second mixture from step (iv) with silica to obtain a second tenofovir disoproxil preparation.

Embodiment 8 is the method of embodiment 7, wherein the first mixture is dried by contacting with a drying agent or by azeotropic distillation.

Embodiment 8a is the method of embodiment 8, wherein the first mixture is dried by azeotropic distillation.

Embodiment 8a(1) is the method of embodiment 8a, wherein a solvent is added to the first mixture prior to the azeotropic distillation, preferably a solvent selected from the group consisting of methanol, ethanol, isopropanol, and dimethylformamide.

Embodiment 8b is the method of embodiment 8, wherein the first mixture is dried by contacting with a drying agent.

Embodiment 8b(1) is the method of embodiment 8b, wherein the drying agent is sodium sulfate, magnesium sulfate, or molecular sieves.

Embodiment 9 is the method of any one of embodiments 7-8b(1), wherein the solvent used in the step (iv) is at least one selected from a group consisted of acetone, methanol, ethanol, 2-propanol, acetonitrile, methyl acetate, ethyl acetate, isopropyl acetate, dichloromethane, toluene, isopropyl ether, diethyl ether, and isopropyl ether.

Embodiment 9a is the method of embodiment 9, wherein the solvent is isopropyl acetate

Embodiment 9b is the method of embodiment 9, wherein the solvent is a mixture of isopropyl acetate/dimethylformamide.

Embodiment 10 is the method of any one of embodiments 7-9b, wherein the step (v) comprises concentrating the second mixture from the step (iv) to obtain a second tenofovir disoproxil preparation.

Embodiment 10a is the method of any one of embodiments 7-9b, wherein the step (v) comprises treating the second mixture from step (iv) with silica to obtain a second tenofovir disoproxil preparation.

Embodiment 10a(1) is the method of embodiment 10a, wherein the silica is standard silica.

Embodiment 10a(2) is the method embodiment 10a, wherein the silica is thiol modified silica (Si-thiol).

Embodiment 11 is the method of any one of embodiments 7-10a(2), further comprising:
(i) reacting the second tenofovir disoproxil preparation with fumaric acid in a solvent to obtain a mixture comprising tenofovir disoproxil fumarate; and
(ii) crystallizing the tenofovir disoproxil fumarate from said mixture to obtain the composition comprising tenofovir disoproxil fumarate and chloromethyl isopropyl carbonate.

Embodiment 12 is the method of embodiment 11, wherein the solvent used in the step (vi) is selected from the group consisting of methanol, ethanol, propanol, and isopropyl alcohol.

Embodiment 12a is the method of embodiment 11, wherein the solvent used in the step (vi) is isopropyl alcohol.

Embodiment 13 is a composition comprising tenofovir disoproxil fumarate, prepared by a method comprising:
(i) reacting tenofovir disoproxil fumarate in a solvent with a base to obtain a first tenofovir disoproxil preparation, or alternatively using a prepared crude preparation of tenofovir disoproxil freebase as a first tenofovir disoproxil preparation;
(ii) washing the first tenofovir disoproxil preparation with water to obtain a first mixture;
(iii) drying the first mixture from the step (ii) to obtain a second tenofovir disoproxil preparation;
(iv) adding a solvent to the second tenofovir disoproxil preparation to obtain a second mixture;
(v) concentrating the second mixture from the step (iv) to obtain a third tenofovir disoproxil preparation; or treating the second mixture from step (iv) with silica to obtain a third tenofovir disoproxil preparation;
(vi) reacting the third tenofovir disoproxil preparation with fumaric acid in a solvent to obtain a mixture comprising tenofovir disoproxil fumarate; and
(vii) crystallizing the tenofovir disoproxil fumarate from said mixture to obtain the composition.

Embodiment 14a is the composition of embodiment 13, wherein a content of chloromethyl isopropyl carbonate in the composition is 50 ppm or less, as measured by gas chromatography (GC).

Embodiment 14b is the composition of embodiment 13, wherein a content of chloromethyl isopropyl carbonate in the composition is 25 ppm or less, as measured by gas chromatography (GC).

Embodiment 14c is the composition of embodiment 13, wherein a content of chloromethyl isopropyl carbonate in the composition is 10 ppm or less, as measured by gas chromatography (GC).

Embodiment 14d is the composition of any one of embodiments 13-14c, wherein a content of chloromethyl isopropyl carbonate is determined by analyzing a solution of the composition by direct injection gas chromatography (GC).

Embodiment 15 is the composition of any one of embodiments 13-14c, wherein the solvent used in the step (i) is selected from the group consisting of acetone, methanol, ethanol, 2-propanol, acetonitrile, methyl acetate, ethyl acetate, isopropyl acetate, dichloromethane, toluene, isopropyl ether, diethyl ether, and isopropyl ether.

Embodiment 15a is the composition of any one of embodiments 13-14c, wherein the solvent used in the step (i) is a mixed solvent of at least one of methyl acetate, ethyl acetate, and isopropyl acetate with water.

Embodiment 15b is the composition of any one of embodiments 13-14c, wherein the solvent used in the step (i) is isopropyl acetate or a mixture of isopropyl acetate and water.

Embodiment 16 is the composition of any one of embodiments 13-15b, wherein the base is an organic base, preferably selected from the group consisting of triethylamine, diethylamine, N,N-diisopropylethylamine, aminomethyl propanol, and ethanolamine.

Embodiment 16a is the composition of any one of embodiments 13-15b, wherein the base is an inorganic base, preferably selected from the group consisting sodium hydroxide, potassium hydroxide, sodium carbonate, sodium bicarbonate, potassium carbonate, potassium bicarbonate, cesium carbonate, cesium bicarbonate, ammonium hydroxide, and ammonium acetate.

Embodiment 16b is the composition of embodiment 16, wherein the base is ethanolamine.

Embodiment 16c is the composition of embodiment 16a, wherein the base is sodium bicarbonate.

Embodiment 17 is the composition of any one of embodiments 13-16c, wherein the first mixture is dried in the step (iii) by contacting with a drying agent.

Embodiment 17a is the composition of any one of embodiments 13-16c, wherein the first mixture is dried in the step (iii) by azeotropic distillation.

Embodiment 17a(1) is the composition of embodiment 17a, wherein a solvent is added to the first mixture prior to the azeotropic distillation, preferably a solvent selected from the group consisting of methanol, ethanol, isopropanol, and dimethylformamide.

Embodiment 18 is the composition of any one of embodiments 13-1a(1), wherein the solvent used in the step (iv) is at least one selected from a group consisted of acetone, methanol, ethanol, 2-propanol, acetonitrile, methyl acetate, ethyl acetate, isopropyl acetate, dichloromethane, isopropyl ether, diethyl ether, and isopropyl ether.

Embodiment 18a is the composition of embodiment 18, wherein the solvent is isopropyl acetate.

Embodiment 18b is the composition of embodiment 18, wherein the solvent is a mixture of isopropyl acetate and dimethylformamide.

Embodiment 19 is the composition of any one of embodiments 13-18b, wherein the step (v) comprises concentrating the second mixture from the step (iv) to obtain a third tenofovir disoproxil preparation.

Embodiment 19a is the composition of any one of embodiments 13-18b, wherein the step (v) comprises treating the second mixture from step (iv) with silica to obtain a third tenofovir disoproxil preparation.

Embodiment 19a(1) is the composition of embodiment 19a, wherein the silica is standard silica.

Embodiment 19a(1) is the composition of embodiment 19a, wherein the silica is thiol modified silica.

Embodiment 20 is the composition of any one of embodiments 13-19a(2), wherein the solvent used in the step (vi) is selected from the group consisting of methanol, ethanol, propanol, and isopropyl alcohol.

Embodiment 20a is the composition of any one of embodiments 13-19a(2), wherein the solvent used in the step (vi) is isopropyl alcohol.

Embodiment 21 is a gas chromatography (GC) method of determining a content of chloromethyl isopropyl carbonate in a composition comprising tenofovir disoproxil fumarate and chloromethyl isopropyl carbonate, the method comprising:
(i) dissolving the composition in a suitable solvent to prepare a sample solution;
(ii) dissolving a reference standard of chloromethyl isopropyl carbonate in a solvent to prepare a standard solution;
(iii) analyzing the sample solution and the standard solution by gas chromatography (GC);
(iv) measuring an area of each GC peak;
(v) determining the content of chloromethyl isopropyl carbonate based on the area from step (iv).

Embodiment 21a is the gas chromatography (GC) method of embodiment 21, wherein the gas chromatography is direct injection gas chromatography.

Embodiment 21b is the gas chromatography (GC) method of any one of embodiments 21-21a, wherein a 95% dimethyl/5% diphenyl polysiloxane or 6% cyanopropylphenyl-94% dimethylpolysiloxane capillary gas chromatography column is used.

Embodiment 21c is the gas chromatography (GC) method of any one of embodiments 21-21b, wherein the gas chromatography comprises flame ionization detection.

Embodiment 21d is the gas chromatography (GC) method of any one of embodiments 21-21c, wherein the solvent in step (i) and step (ii) is dimethylformamide (DMF).

Embodiment 21e is the gas chromatography (GC) method of any one of embodiments 21-21d, wherein the sample solution has a concentration of about 200 mg/mL.

The following examples are to further illustrate the nature of the invention. It should be understood that the following examples do not limit the invention and that the scope of the invention is determined by the appended claims.

### REFERENCES

1. U.S. Pat. No. 5,922,695
2. US 2013/0005969 A1
3. WO 2008007392 A2
4. USP Pending Monography (v. 1, 2011)

### EXAMPLES

The following abbreviations and chemical notations are used in the following examples, unless clearly stated otherwise:

IPE: isopropyl ether
IPAc: isopropyl acetate
IPA: isopropyl alcohol
DMF: dimethylformamide
DCM: methylene chloride or dichloromethane
ACN: acetonitrile
LAH: lithium aluminum hydride
HBr: hydrobromic acid
NH₄OH: ammonium hydroxide
HSGC: headspace gas chromatography
HPLC: high performance liquid chromatography
¹H NMR: proton nuclear magnetic resonance
¹³C NMR: carbon nuclear magnetic resonance
m.p.: melting point

### Example 1: Purification Process to Reduce the Content of Chloromethyl Isopropyl Carbonate (CMIC) from Preparations of Tenofovir Disoproxil Fumarate (TDF)

Tenofovir disoproxil fumarate (TDF, 600 g) (CMIC content=206 ppm) was suspended in IPAc (7.34 kg). Water (3.00 kg) and sodium bicarbonate (300 g) were added to this suspension and the resulting mixture was stirred at 25° C. for 30 minutes. Agitation was stopped and the layers were separated for 30 minutes. The bottom aqueous layer was split and additional water (1.20 kg) was added to the organic layer. The resulting mixture was stirred for 30 minutes and then settled for 1 hour, and the bottom aqueous portion was split. Molecular sieves (1.20 kg) and IPA (1.20 kg) were charged to the organic layer and the resulting mixture was stirred at 25° C. for 16 hours to remove water. The molecular sieves were removed by filtration and washed with IPAc (600 g). The organic solution was polish filtered into the reactor, and silica modified with a thiol (Si-thiol, 300 g) was charged to the filtrate. The slurry was stirred at 25° C. for 2 hours and the Si-thiol was filtered and rinsed with IPAc (600 g). The solvent was removed under vacuum at 25-30° C. to provide tenofovir disoproxil as a white solid.

Isopropanol (4.20 kg) was charged to the tenofovir disoproxil solid and the mixture was heated to 50° C. to dissolve. Fumaric acid (254 g) was charged to the solution and the mixture was heated to 70° C. to dissolve. The solution was cooled to about 40° C. and seeded with a small amount of TDF to initiate crystallization. The resulting slurry was cooled to about 20° C. for approximate 5 hours and stirred for an additional 8-10 hours. The slurry was filtered and washed three times with isopropyl ether (3×2.35 kg). The isolated material was dried under vacuum to provide 495 g TDF (82.5% yield, CMIC content=14 ppm). Residual CMIC in the isolated TDF product and starting TDF was determined by direct injection GC.

### Example 2: Purification Process to Reduce the Content of Chloromethyl Isopropyl Carbonate (CMIC) from Preparations of Tenofovir Disoproxil Fumarate (TDF)

The starting material, tenofovir disoproxil fumarate, was determined to contain 474 ppm of CMIC. TDF (22 g) was suspended in IPAc (310 ml) at 22° C. A solution of sodium bicarbonate (10.8 g) in water (110 mL) was added to the suspension, and the mixture was stirred to provide a homogeneous biphasic mixture. The mixture was stirred for an additional 30 minutes, and then agitation was stopped. The layers were separated for 30 minutes, and the bottom aqueous layer was split. Water (44 mL) was charged to the organic layer and the resulting mixture was stirred for 30 minutes. After the agitation was stopped, the layers were separated for 1 hour, and the bottom aqueous layer was split. Water was then removed from the organic phase by distillation under vacuum at 25° C.-30° C. to provide a white solid. The solid material was reconstituted in a mixture of IPAc (250 ml) and IPA (55 ml), and the resulting mixture was heated to 50° C. to form a solution, and then cooled to 25° C. Silica modified with a thiol (Si-thiol, 11.0 g) was added to the solution, and the mixture was stirred at 25° C. for 2 hours. The mixture was filtered to remove Si-thiol and rinsed with IPAc (19 g). The organic layer was distilled under vacuum at 25° C.-30° C. to provide a tenofovir disoproxil as a white solid.

Isopropyl alcohol (154 g) was charged to the tenofovir disoproxil solid, and the mixture was heated to 50° C. to dissolve the solid. Fumaric acid (9.36 g, 2.33 mol eq.) was charged and the resulting solution was heated to 70° C. to give a homogenous solution. The solution was then cooled to about 40° C. and seeded with a small amount of TDF to induce crystallization. Once crystallization occurred, the slurry was cooled to about 20° C. over 5 hours and stirred at about 20° C. for at least 2 hours. The slurry was filtered, and the filter cake was washed with IPE (100-150 ml). The filter cake was slurry washed with about 100 ml of IPE, filtered, and then washed with about 125 ml of IPE. The isolated product was dried under vacuum for 18 hours at 45° C., which afforded TDF. The isolated yield of TDF was 18.7 g (85.0% recovery, CMIC content=15 ppm). Residual CMIC in the isolated TDF product and starting TDF was determined by direct injection gas chromatography as described below in Example 3.

### Example 3: Quantification of CMIC in TDF Preparations Determined by Direct Injection Gas Chromatography

Samples were prepared by dissolving TDF in DMF to prepare a solution with the concentration of approximate 200 mg/mL. The sample solutions were analyzed by direct injection gas chromatography with flame ionization detection. The column used was a 95% dimethyl/5% diphenyl polysiloxane capillary gas chromatography column. The inlet temperature was optimized at 160° C. to exceed the boiling point of CMIC (147.5° C.). Quantitative analysis was performed using external standard of CMIC prepared in DMF at 10 ppm or 50 ppm relative to the TDF sample concentration. Acceptable sensitivity has been demonstrated at 10 ppm, with signal to noise ratio determined as greater than 10.

The resulting peak area of the CMIC in the TDF sample solution is used to calculate the CMIC content in ppm based on the response relative to an external standard of CMIC. Specifically, the CMIC content (ppm) is calculate as follows:

$$CMIC ( ppm ) = \frac{{A}_{Sam}}{{A}_{Std}} \times \frac{{C}_{Std}}{{C}_{Sam}} \times 1000000$$

where:
Aₛₐₘ: peak area of CMIC in the sample solution
A_{std}: average peak area of CMIC in the Standard Solutions
C_{Sam}: Sample Concentration (mg/mL)
C_{Std}: Working Standard CMIC Concentration (mg/mL)

### Example 4: Solvent Reslurry Screen to Remove Chloromethyl Isopropyl Carbonate (CMIC)

Tenofovir disoproxil fumarate (TDF) solid (1 g) was charged to multiple vials containing 10 mL of solvent at ambient temperature for at least 1 hour to prepare a slurry. Then, each slurry was filtered and washed with 10 mL of isopropyl ether (IPE). The resulting solids were dried at 28° C. overnight and analyzed by headspace gas chromatography (HSGC) to determine the CMIC content. The analysis data are shown in Table 1 below.

TABLE 1

| | | CMIC Content | |
|---|---|---|---|
| Sample # | Solvent | (ppm) | % Recovery |
| Initial | N/A | 115 | NA |
| 1 | Acetone | 29 | 66.02 |
| 2 | 2-Propanol | 42 | 77.88 |
| 3 | Acetonitrile | 24 | 87.38 |
| 4 | isopropyl acetate | 24 | 92.16 |
| 5 | Dichloromethane | 68 | 92.16 |
| 6 | Isopropyl ether | 33 | 96.15 |
| 7 | Diethyl ether | 56 | 95.15 |

The data showed that the slurry in different solvents can reduce the content of CMIC in TDF.

### Example 5: Isopropyl Acetate (IPAc) Iterative Co-Distillation to Remove CMIC from TDF Preparations

Solid TDF (10 g) was mixed with 100 mL of IPAc to form a suspension. Then, the following iterative co-distillation process was performed: (a) the suspension was distilled under vacuum by rotovap to a wet solid (bath temp at 28° C.); (b) the wet solid was reconstituted to the initial starting volume with IPAc to obtain a bulk suspension; and (c) a sample was pulled from the bulk suspension, filtered, and washed with 10 mL of IPE (iteration #1).

The remainder of the bulk suspension was carried through steps a-c to generate iteration #2-4. The resulting solids were dried at 28° C. overnight and analyzed by HSGC for CMIC content. The analysis data are shown in Table 2 below.

TABLE 2

| | | CMIC Content |
|---|---|---|
| Sample # | Iteration No. | (ppm) |
| Initial | N/A | 115 |
| 1 | 1 | 43 |
| 2 | 2 | 38 |
| 3 | 3 | 53 |
| 4 | 4 | 50 |

The data showed that co-distillation with IPAc can reduce the content of CMIC in DMF, but the iteration does not typically significantly further improve the purity.

### Example 6: NMR Study of CMIC Degradation in TDF Preparations

A fixed quantity (0.60 ml) of CMIC was charged to multiple vials containing a fixed quantity (3.00 ml) of IPAc. Then various reagents were added to the vials and the resulting biphasic mixtures were agitated. After phase separation, the upper organic layer was sampled for analysis of the amount of CMIC relative to the amount of IPAc. The analysis was performed by ¹H NMR in d₆-DMSO. The larger the IPAc signal detected, the more efficient the reagent was at reacting with CMIC. The results are summarized in Table 3 below:

TABLE 3

| | | CMIC | IPAc |
|---|---|---|---|
| Sample # | Tested Base | Integration | Integration |
| 1 | Control Trial (N/A) | 1.00 | 8.19 |
| 2 | 5% Sodium Bicarbonate | 1.00 | 8.10 |
| 3 | 5% Sodium Carbonate | 1.00 | 7.99 |
| 4 | 10% Ammonium Hydroxide | 1.00 | 10.53 |
| 5 | 10% Ammonium Acetate | 1.00 | 7.90 |
| 6 | Diethylamine | 1.00 | 16.69 |
| 7 | Ethanolamine | 1.00 | 5788.30 |
| 8 | 10% Hydrochloric Acid | 1.00 | 7.78 |
| 9 | 10% Sodium Hydroxide | 1.00 | 7.70 |
| 10 | Diethylamine + 1 mL Water Spike | 1.00 | 16.34 |
| | (First Intent Exp.) | | |
| 11 | Ethanolamine + 1 mL Water Spike | 1.00 | 108.61 |
| | (First Intent Exp.) | | |

The data showed that a variety of bases that could be used to convert TDF to freebase TD also react with CMIC.

### Example 7: Purification Process to Reduce the Content of CMIC in TDF Preparations

A 40 g sample of TDF (CMIC content=2307 ppm) was taken up in IPAc (490.35 g) at 22° C., and then treated with aqueous NaHCO₃ (prepared with 16.35 g of NaHCO₃ and 196.14 g of H₂O). The resulting mixture was held at room temperature for 1 h, resulting in a clear, biphasic solution. After one additional water wash (65.38 g) of the organic layer, DMF (26.81 g) was charged and the resulting mixture was stirred for 20 minutes. The resulting solution was dried azeotropically under vacuum at 20° C. to afford an oil, and the oil was reconstituted with IPAc (490.35 g) to form a solution. The resulting solution was polish filtered to remove any residual particulates. A second vacuum distillation was performed at 20-28° C., affording tenofovir disoproxil as a white solid.

Fumaric acid (17.00 g) and IPA (278.85 g) were charged to a separate reactor and the resulting slurry was heated to 70° C. To the hot fumaric acid/IPA slurry was charged the isolated tenofovir disoproxil, and the resulting mixture was allowed to be re-heat to 70° C. until all solids dissolved. Once complete dissolution was achieved, the mixture was cooled to 40° C., and seeded at 42° C. The resulting crystallized slurry was ramped from 40° C. to about 20-24° C. for approximate five hours and allowed to age at overnight. The TDF product was filtered and displacement washed with about 120-140 ml of IPE, and the slurry was washed with IPE (156.91 g) and displacement washed a few times with IPE, each time with about 120-140 ml. The final product was dried under vacuum at no higher than 45° C. (target 28° C.) overnight, which afforded 36.09 g of TDF with 90.23% recovery, containing 29 ppm CMIC as determined by headspace gas chromatography (HSGC).

### Example 8: Purification Process to Remove CMIC from TDF

A 40 g sample of TDF (CMIC content=2307 ppm) was taken up in IPAc (490.35 g) at 22° C., and treated with aqueous sodium bicarbonate (prepared with 16.35 g of NaHCO₃ and 196.14 g of H₂O). The resulting mixture was allowed to age 1 h, resulting in a clear, biphasic solution. After one additional water wash (65.38 g) of the organic layer, DMF (26.81 g) was charged and the resulting mixture was allowed to stir for 20 minutes. The resulting solution was dried azeotropically under vacuum at 20° C. to afford an oil, and the oil was reconstituted with IPAc (490.35 g) to form a solution. The resulting solution was polish filtered to remove any residual particulates. To the solution was charged an additional portion of DMF (26.81 g) to assist is solvating TD and followed by silica treatment performed by addition of silica (10.13 g) to the organics, stirring, and filtration. After filtering off the silica, a second vacuum distillation was performed at 20-28° C., affording an oily TD slurry.

To a separate reactor were charged fumaric acid (17.00 g) and IPA (278.85 g), and the resulting slurry was heated to 70° C. To the hot fumaric acid/IPA slurry was charged the isolated TD, and the resulting mixture was allowed to re-heat to 70° C. until all solids dissolved. Once the complete dissolution was achieved the mixture was cooled to 40° C., and seeded at 42° C. The resulting crystallized slurry was ramped from 40° C. to about 20-24° C. for approximate five hours, and allowed to age overnight. The TDF product was filtered and displacement washed with about 120-140 ml of IPE, a few times, each time with about 120-140 ml. The final product was dried under vacuum at no more than 45° C. (target 28° C.) overnight, which afforded 32.42 g of TDF with 81.05% recovery, containing 21 ppm CMIC as determined by HSGC.

### Example 9: Purification Process to Remove CMIC from TDF

A 40 g sample of TDF (CMIC content=2307 ppm) was taken up in IPAc (490.35 g)/water (196.14 g) at 22° C., and treated with ethanolamine (11.89 g). The resulting mixture was allowed to age 1 h (i.e., stand at room temperature for 1 h), resulting in a biphasic solution. After one additional water wash (65.38 g) of the organic layer, DMF (26.81 g, 0.82 S) was charged and allowed to stir for 20 minutes. The resulting solution was dried azeotropically under vacuum at 20° C. to afford an oil, and the oil was reconstituted with IPAc (490.35 g) to form a solution. The resulting solution was polish filtered to remove any residual particulates. A second vacuum distillation was performed at 20-28° C., affording a wet, oily, sticky, and white TD solid.

Fumaric acid (17.00 g) and IPA (278.85 g) were charged to a separate reactor and the resulting slurry was heated to 70° C. To the hot fumaric acid/IPA slurry was charged the isolated TD, and the mixture was allowed to re-heat to 70° C. until all solids had dissolved. Once the complete dissolution was achieved the mixture was cooled to 40° C., and seeded at 42° C. The resulting crystallized slurry was ramped from 40° C. to about 20-24° C. for approximate five hours and allowed to age overnight. The TDF product was filtered and displacement washed with about 120-140 ml of IPE a few times, each time with about 120-140 ml. The final product was dried under vacuum at no more than 45° C. (target 28° C.) overnight, which afforded 30.15 g of TDF with 75.38% recovery, containing 18 ppm CMIC as determined by HSGC.

### Example 10: Purification Process to Remove CMIC from TDF

A 40 g sample of TDF (CMIC content=2307 ppm) was taken up in IPAc (490.35 g)/water (196.14 g) at 22° C., and treated with ethanolamine (11.89 g). The resulting mixture was allowed to age 1 h, resulting in a biphasic solution. After one additional water wash (65.38 g) of the organic layer, DMF (26.81 g, 0.82 S) was charged and allowed to stir for 20 minutes. The resulting solution was dried azeotropically under vacuum at 20° C. to afford an oil, and the oil was reconstituted with IPAc (490.35 g) to form a solution. The resulting solution was polish filtered to remove any residual particulates and followed by silica treatment performed by addition of silica (10.13 g) to the organics, stirring, and filtration. After filtering off the silica, a second vacuum distillation was performed at 20-28° C., affording a slightly sticky, wet, and white TD solid.

Fumaric acid (17.00 g) and IPA (278.85 g) were charged to a separate reactor and the resulting slurry was heated to 70° C. To the hot fumaric acid/IPA slurry was charged the isolated TD, and the mixture was allowed to re-heat to 70° C. until all solids had dissolved. Once the complete dissolution was achieved the mixture was cooled to 40° C., and seeded at 42° C. The resulting crystallized slurry was ramped from 40° C. to 22° C. over five hours, and allowed to age overnight. The TDF product was filtered and displacement washed with IPE (156.91 g), slurry washed with IPE (156.91 g) and displacement washed with IPE (156.91 g). The final product was dried under vacuum at no more than 45° C. (target 28° C.) overnight, which afforded 28.18 g of TDF with 70.45% recovery, containing 21 ppm CMIC as determined by HSGC.

### Example 11: Purification Process to Remove CMIC from TDF

A 40 g sample of TDF (CMIC content=2307 ppm) was taken up in IPAc (490.35 g) at 22° C., and treated with aqueous sodium bicarbonate (prepared with 16.35 g of NaHCO₃ and 196.14 g of H₂O). After one additional water wash (65.38 g) of the organic layer, DMF (26.81 g, 0.82 S) was charged and allowed to stir for 20 minutes. The resulting solution was dried azeotropically under vacuum at 20° C. to afford an oil, and the oil was reconstituted with IPAc (490.35 g) to form a solution. The resulting solution was polish filtered to remove any residual particulates and followed by silica treatment performed by addition of silica (10.13 g) to the organics, stirring, and filtration. After filtering off the silica, a second vacuum distillation was performed at 20-28° C., affording a white and slightly wet TD solid.

Fumaric acid (17.00 g) and IPA (278.85 g) were charged to a separate reactor and the resulting slurry was heated to 70° C. To the hot fumaric acid/IPA slurry was charged the isolated TD, and the mixture was allowed to re-heat to 70° C. until all solids had dissolved. Once the complete dissolution was achieved the mixture was cooled to 40° C., and seeded at 42° C. The resulting crystallized slurry was ramped from 40° C. to 22° C. over five hours and allowed to age overnight. The TDF product was filtered and displacement washed with IPE (156.91 g), slurry washed with IPE (156.91 g) and displacement washed with IPE (156.91 g). The final product was dried under vacuum at no more than 45° C. (target 28° C.) overnight, which afforded 34.18 g of TDF with 85.45% recovery, containing 63 ppm CMIC as determined by HSGC.

### Example 12: Purification Process to Remove CMIC from TDF

A 40 g sample of TDF (CMIC content=894 ppm) was taken up in IPAc (490.35 g)/water (196.14 g) at 22° C., and treated with 28% ammonium hydroxide (24.36 g). The resulting mixture was allowed to age 1 h, resulting in a biphasic solution. After one additional water wash (65.38 g) of the organic layer, DMF (26.81 g) was charged and allowed to stir for 20 minutes. The resulting solution was dried azeotropically under vacuum at 20° C. to afford an oil, and the oil was reconstituted with IPAc (490.35 g) to form a solution. The resulting solution was polish filtered to remove any residual particulates and followed by silica treatment performed by addition of silica (10.13 g) to the organics, stirring, and filtration. After filtering off the silica, a second vacuum distillation was performed at 20-28° C., affording a white and oily TD solid.

Fumaric acid (17.00 g) and IPA (278.85 g) were charged to a separate reactor and the resulting slurry was heated to 70° C. To the hot fumaric acid/IPA slurry was charged the isolated TD, and the mixture was allowed to re-heat to 70° C. until all solids had dissolved. Once the complete dissolution was achieved the mixture was cooled to 40° C., and seeded at 42° C. The resulting crystallized slurry was ramped from 40° C. to about 20-24° C. for approximate five hours, and allowed to age overnight. The TDF product was filtered and displacement washed with about 120-140 ml IPE, a few times, each time with about 120-140 ml. The final product was dried under vacuum at no more than 45° C. (target 28° C.) overnight, which afforded 21.21 g of TDF with 53.03% recovery, containing 89 ppm CMIC as determined by direct injection gas chromatography.

### Example 13: Study on Removal of CMIC from TDF Via Methanol Co-Distillation

A total of 60 grams of TDF was dissolved in methanol (715 g) at room temperature. To the solution was charged 0.4 wt. % CMIC (0.240 g), and allowed to stir for approximately 10 minutes. The solvent was subsequently removed by vacuum distillation via rotary evaporation at 20-28° C., and the product dried under vacuum at 28° C. It afforded 59.97 g of TDF with 99.88% recovery, containing 184 ppm CMIC by HSGC. A total of five subsequent iterations are currently in progress, whereby the isolated TDF from the preceding trail was re-dissolved in methanol (no additional CMIC spike), vacuum distilled to a solid, and dried at 28° C. The results will identify whether the CMIC level in the resulting TDF trends downward throughout each iteration. Similar to Example 5, the distillations with methanol reduced CMIC content from 184 ppm to less than 100 ppm. Specifically, the residual CMIC content was 102 ppm after the first distillation, 126 ppm after the second distillation, and 72 ppm after the third distillation

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A composition comprising tenofovir disoproxil fumarate, wherein a content of chloromethyl isopropyl carbonate in the composition, as measured by analyzing a solution of the composition by direct injection gas chromatography (GC), is about 50 ppm or less.

2. The composition of claim 1, wherein the content of chloromethyl isopropyl carbonate is about 25 ppm or less.

3. The composition of claim 1, wherein the content of chloromethyl isopropyl carbonate is about 10 ppm or less.

4. A pharmaceutical composition comprising at least one pharmaceutically acceptable carrier and the composition of claim 1.

5. A method of preparing a composition comprising tenofovir disoproxil fumarate, wherein a content of chloromethyl isopropyl carbonate in the composition, as measured by analyzing a solution of the composition by direct injection gas chromatography (GC), is about 50 ppm or less, the method comprising:
(i) reacting tenofovir disoproxil fumarate with a base to obtain a first tenofovir disoproxil preparation;
(ii) washing the first tenofovir disoproxil preparation with water to obtain a first mixture, and drying the first mixture to obtain a second tenofovir disoproxil preparation;
(iii) diluting the second tenofovir disoproxil preparation with an organic solvent to obtain a second mixture, and adding silica to the second mixture to obtain a third mixture;
(iv) removing the silica from the third mixture and concentrating the filtered third mixture to obtain a third tenofovir disoproxil preparation;
(v) reacting the third tenofovir disoproxil preparation with fumaric acid in a solvent to obtain a fourth mixture comprising tenofovir disoproxil fumarate; and
(vi) crystallizing the tenofovir disoproxil fumarate from the fourth mixture to obtain the composition.

6. The method of claim 5, wherein the solvent in steps (i) and (iii) is independently selected from the group consisting of isopropyl acetate and a mixture of isopropyl acetate and water.

7. The method of claim 5, wherein the base in step (i) is selected from the group consisting of sodium bicarbonate, sodium carbonate, sodium hydroxide, ammonium hydroxide, ammonium hydroxide, ammonium acetate, diethyl amine, and ethanolamine.

8. The method of claim 7, wherein the base is sodium bicarbonate or potassium bicarbonate.

9. The method of claim 5, wherein the first mixture is dried by contacting with a drying agent or by azeotropic distillation.

10. The method of claim 5, wherein the silica in step (iii) is thiol-modified silica.

11. The method of claim 5, wherein the solvent in step (v) is isopropyl alcohol.

12. A composition comprising tenofovir disoproxil fumarate, wherein a content of chloromethyl isopropyl carbonate in the composition, as measured by analyzing a solution of the composition by direct injection gas chromatography (GC), is about 50 ppm or less, the composition prepared by the method of claim 5.

13. A method for quantifying a content of chloromethyl isopropyl carbonate in a composition comprising tenofovir disoproxil fumarate (TDF), the method comprising:
(i) dissolving the composition in a solvent to prepare a sample solution;
(ii) dissolving a reference standard of chloromethyl isopropyl carbonate (CMIC) in a solvent to prepare a standard solution
(iii) analyzing the sample solution and standard solution by direct injection gas chromatography;
(iv) identifying a peak corresponding to CMIC for each of the sample solution and the standard solution from step (iii); and
(v) quantifying the content of CMIC in the composition by comparing an area of the peak corresponding to CMIC in the sample solution to an area of the peak corresponding to CMIC in the standard solution.

14. The method of claim 13, wherein the solvent in step (i) and step (ii) is dimethylformamide (DMF).

15. The method of claim 13, wherein the sample solution comprises TDF at a concentration of about 200 mg/mL.

16. The method of claim 5, further comprising quantifying a content of chloromethyl isopropyl carbonate (CMIC) in the composition by a method comprising:
(i) dissolving the composition in a solvent to prepare a sample solution;
(ii) dissolving a reference standard of CMIC in a solvent to prepare a standard solution
(iii) analyzing the sample solution and standard solution by direct injection gas chromatography;
(iv) identifying a peak corresponding to CMIC for each of the sample solution and the standard solution from step (iii); and
quantifying the content of CMIC in the composition by comparing an area of the peak corresponding to CMIC in the sample solution to an area of the peak corresponding to CMIC in the standard solution.
